(19) United States
(12) Patent Application Publication
(10) Pub. No.: US 2024/0417504 A1
(43) Pub. Date: Dec. 19, 2024

(54) VEGETABLE OIL POLYOL, PREPARATION METHOD THEREFOR, AND USE THEREOF

(71) Applicants: NANJING TECH UNIVERSITY, Nanjing (CN); CHINA CONSTRUCTION INDUSTRIAL & ENERGY ENGINEERING GROUP CO., LTD., Nanjing (CN)

(72) Inventors: Kai GUO, Nanjing (CN); Zheng FANG, Nanjing (CN); Yiping HUANG, Nanjing (CN); Wei HE, Nanjing (CN); Hong QIN, Nanjing (CN); Changhai YUE, Nanjing (CN); Chengkou LIU, Nanjing (CN); Fujian LIU, Nanjing (CN); Jindian DUAN, Nanjing (CN); Jingjing HUANG, Nanjing (CN); Jiangkai QIU, Nanjing (CN)

(73) Assignees: NANJING TECH UNIVERSITY, Nanjing (CN); CHINA CONSTRUCTION INDUSTRIAL & ENERGY ENGINEERING GROUP CO., LTD., Nanjing (CN)

(21) Appl. No.: 18/819,162

(22) Filed: Aug. 29, 2024

(30) Foreign Application Priority Data
Sep. 20, 2023 (CN) 2023112216701

(51) Int. Cl. C08G 18/36; C08G 18/18

(52) U.S. Cl. CPC C08G 18/36; C08G 18/18; C08G 2150/90

(57) ABSTRACT

An epoxidized vegetable oil is subjected to a first ring-opening reaction with an acidic catalyst and a β-mercaptoalcohol compound to obtain a first reaction solution, and then the first reaction solution is subjected to a second ring-opening reaction with a cyclohydrocarbyl methanol compound to obtain the vegetable oil polyol, which adopts novel ring-opening reagents to introduce an antioxidant sulfur-containing fragment into the molecular structure of the vegetable oil polyol in a covalent manner, while also introducing a cyclohydrocarbyl group and retaining a small portion of epoxy groups, thereby ensuring that a polyurethane product has a certain degree of toughness, in addition to relatively good corrosion resistance and oxidation resistance, while guaranteeing the mechanical properties of the polyurethane material.

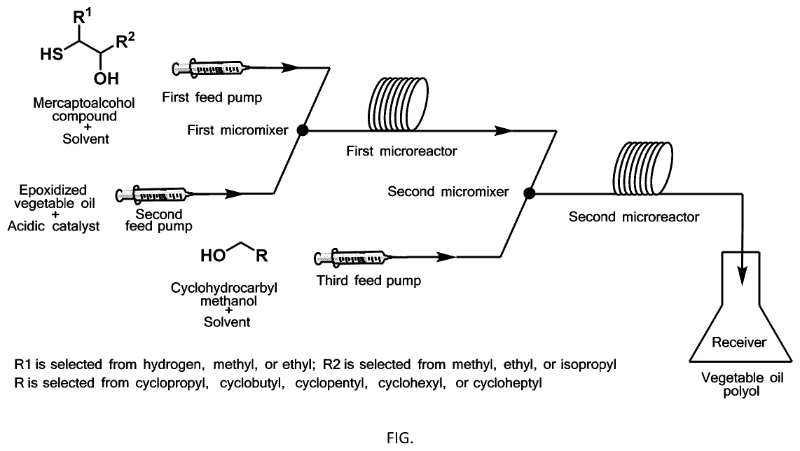

### CROSS-REFERENCE TO RELATED APPLICATIONS

This application claims priority to Chinese Patent Application Ser. No. CN2023112216701 filed on 20 Sep. 2023.

### TECHNICAL FIELD

The present invention belongs to the technical field of chemical materials and production thereof, and relates to a vegetable oil polyol, a preparation method therefor, and use thereof.

### BACKGROUND

Polyurethanes are polymers with repeating structural units of urethane chain segments made from the reaction of isocyanates with polyols. Polyurethane articles are divided into two main categories: foamed articles and non-foamed articles. The foamed articles include soft, hard, and semi-hard polyurethane foam plastics; the non-foamed articles include coating materials, adhesives, synthetic leathers, elastomers, elastic fibers, and the like. Among them, the polyurethane coating materials are used in a wide range of applications and in huge quantities. The development of bio-based polyurethane coating materials is currently attracting a great deal of attention from researchers, scholars, and businesses.

The development of vegetable oil polyols is considered an effective approach for the advancement of bio-based materials. As an important monomer for bio-based polyurethane materials, the vegetable oil polyols are derived from vegetable oils as starting materials through chemical modification of their molecular structures, making them an important renewable resource. They react with isocyanate compounds to generate polyurethanes and serve as a good substitute starting material for petroleum-based polyols. Additionally, they are a breakthrough in the development of bio-based polyurethane coating materials. The scheme of oxidizing a vegetable oil into an epoxidized vegetable oil, followed by reactions such as ring-opening to generate a polyhydroxy compound, is highly atom-economical. Additionally, it has the advantages of flexibility, structural controllability, and molecular diversity, making it the main method for developing bio-based polyols at present.

Long-chain groups in the structure of vegetable oil replace the repeating units of traditional petrochemical polyether or polyester polyols, and the basic parent nucleus of triglyceride in the structure has a star-shaped spatial conformation, which imparts more functional properties and application space to downstream polyurethanes. However, vegetable oil polyols often have performance deficiencies, mainly due to their relatively high functionality and uncontrollable reaction processes. During the conversion of functional groups, multiple epoxy groups and ester groups tend to participate in multiple side reaction processes, making it difficult to construct the designed molecular structure through traditional chemical methods. This greatly limits the quality of the polyols, often requiring them to be mixed with traditional petrochemical polyols to achieve a certain application effect. Thus, developing coating material products capable of fully replacing petrochemical polyol poses great challenges.

In addition, coating materials are used in a wide range of applications, which have different requirements for the performance of coating material products, but often the high level of oxidation resistance is the common demand. Typically, antioxidants are added into coating material formulations, but adding a small amount of antioxidants makes it difficult to meet the durability requirements, and adding an excessive amount of antioxidants directly affects the performance of the coating materials in various aspects. Therefore, through a polyol molecular structure derivatization scheme, a fragment with oxidation resistance is introduced into the molecular structure, and is incorporated into the polymer material by means of covalent bonding, avoiding the influence of small molecule additives on the material performance and achieving modification from the material's essence. Since sulfur-containing compounds have certain antioxidant effects, introducing a sulfur-containing group and imparting a polyhydroxy functional group in the structure is an effective scheme for the development of bio-based polyols.

However, due to the relatively higher functionality, it is difficult to effectively control the reaction process by using the conventional reaction scheme of epoxy ring opening. Analysis of the reaction mechanism reveals that the main reason is the poor miscibility between the oil ester and the reaction reagents, along with relatively low reactivity, which requires a long-time and high-intensity reaction. However, the influence of multiple functional groups in the structure makes it difficult to balance reaction selectivity and conversion rate, so that the process controllability is poor, and thus the obtained product has poor molecular uniformity, high viscosity, and relatively significant differences between the macroscopic indexes and the microscopic indexes of individual molecules. Thus, even though vegetable oils are often cheaper than monomers of petrochemical repeating units, it is difficult to obtain vegetable oil polyol products that are superior in both cost and quality. It is necessary to control product quality through chemical process control. In this reaction system, using microreaction technology to intensify the chemical reaction process and achieve continuous and precise control is an effective scheme. Given the current situation where the vegetable oils have non-uniform components and unclear structure-activity relationships, the quality control of the polyol products can only be achieved through reaction process control and macroscopic index regulation, so that controlling product uniformity as much as possible through process control plays an important role in the development of new polyol products and their downstream applications.

### SUMMARY

The present invention aims to solve the technical problems of the deficiencies in the prior art, and thus provides a vegetable oil polyol, a preparation method therefor, and use thereof.

The idea of present invention is as follows: In order to ensure that bio-based polyurethane coating materials maintain their conventional performance while also having certain antioxidant and anticorrosion effects, in the present invention, a polyfunctional ring-opening reagent having groups such as mercapto and hydroxyl groups is used as a first ring-opening reagent to complete a ring-opening reaction of epoxy groups in a vegetable oil with the mercapto groups through process control; then, a highly active primary alcohol containing a cycloalkane group is used to carry out a ring-opening reaction on the residual, less reactive epoxy groups in the vegetable oil, thereby controlling the partial retention of epoxy groups in a class of vegetable oil polyol products; in addition, in order to avoid cross-linking side reactions where the secondary hydroxyl groups generated in the ring-opening reaction are subjected to a non-selective ring-opening reaction with other epoxy groups, the inventors adopt a microchannel reaction device as a reaction apparatus to further control the ring-opening groups.

In order to solve the technical problems described above, the present invention adopts the following technical schemes:

The present invention discloses a preparation method for a vegetable oil polyol, wherein an epoxidized vegetable oil is subjected to a first ring-opening reaction with an acidic catalyst and a first ring-opening reagent to obtain a first reaction solution, and then the first reaction solution is subjected to a second ring-opening reaction with a second ring-opening reagent to obtain a reaction solution comprising the vegetable oil polyol, wherein

the first ring-opening reagent is a β-mercaptoalcohol compound; the second ring-opening reagent is a cyclohydrocarbyl methanol compound.

In some embodiments, the epoxidized vegetable oil is any one or a combination of two or more of an epoxidized olive oil, an epoxidized peanut oil, an epoxidized rapeseed oil, an epoxidized cottonseed oil, an epoxidized soybean oil, an epoxidized coconut oil, an epoxidized palm oil, an epoxidized sesame oil, an epoxidized corn oil, and an epoxidized sunflower seed oil, preferably an epoxidized cottonseed oil or an epoxidized soybean oil.

In some embodiments, the acidic catalyst is any one or a combination of two or more of fluoroboric acid, concentrated sulfuric acid, p-toluenesulfonic acid, methanesulfonic acid, and benzenesulfonic acid, preferably fluoroboric acid, wherein

the acidic catalyst is present in the form of an aqueous solution.

In some embodiments, a structural formula of the β-mercaptoalcohol compound is

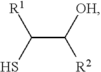

wherein R¹ is selected from hydrogen, methyl, or ethyl; R² is selected from methyl, ethyl, or isopropyl; preferably, R¹ is selected from hydrogen and R² is selected from methyl or ethyl;
when R¹ is selected from hydrogen and R² is selected from methyl, the β-mercaptoalcohol compound is 1-mercapto-2-propanol; when R¹ is selected from hydrogen and R² is selected from ethyl, the β-mercaptoalcohol compound is 1-mercapto-2-butanol.

In some embodiments, a structural formula of the cyclohydrocarbyl methanol compound is

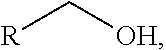

wherein R is selected from cyclopropyl, cyclobutyl, cyclopentyl, cyclohexyl, or cycloheptyl, preferably cyclopropyl, cyclobutyl, cyclopentyl, or cyclohexyl.

In some embodiments, a mass percentage ratio of the epoxidized vegetable oil to the acidic catalyst is 1:(0.02%-0.12%), preferably 1:0.1%.

In some embodiments, a molar ratio of an epoxy group in the epoxidized vegetable oil to the first ring-opening reagent is 1:(0.4-0.7), preferably 1:(0.6-0.7), further preferably 1:0.6.

In some embodiments, a molar ratio of an epoxy group in the epoxidized vegetable oil to the second ring-opening reagent is 1:(0.3-0.6), preferably 1:(0.45-0.6).

In some embodiments, the first ring-opening reaction is carried out at a reaction temperature of 60-100° C., preferably 60-80° C., further preferably 60-70° C., even further preferably 65° C.

In some embodiments, the second ring-opening reaction is carried out at a reaction temperature of 60-100° C., preferably 70-100° C., further preferably 70-90° C., even further preferably 80° C.

In some embodiments, the first ring-opening reaction is carried out in a first organic solvent, and the first organic solvent is any one or a combination of two or more of ethyl acetate, dichloromethane, dichloroethane, chloroform, n-hexane, tetrahydrofuran, 1,4-dioxane, carbon tetrachloride, toluene, and xylene, preferably dichloromethane.

Specifically, a mass-to-volume ratio of the first ring-opening reagent to the first organic solvent is 1 g:0.5-2 mL, preferably 1 g:1-2 mL, further preferably 1 g:1.2-1.6 mL.

In some embodiments, the second ring-opening reaction is carried out in a second organic solvent, and the second organic solvent is any one or a combination of two or more of ethyl acetate, dichloromethane, dichloroethane, chloroform, n-hexane, tetrahydrofuran, 1,4-dioxane, carbon tetrachloride, toluene, and xylene, preferably dichloromethane.

Specifically, a mass-to-volume ratio of the second ring-opening reagent to the second organic solvent is 1 g:1-4 mL, preferably 1 g:1.6-3.6 mL, further preferably 1 g:2.2-3.2 mL.

In some embodiments, the vegetable oil polyol is prepared using a conventional reactor or a microchannel reaction device; preferably, the vegetable oil polyol is prepared using a microchannel reaction device.

In some embodiments, the preparation of the vegetable oil polyol using the microchannel reaction device comprises the following steps:
mixing the epoxidized vegetable oil with the acidic catalyst to obtain a first mixed solution, mixing the first ring-opening reagent with a first organic solvent to obtain a second mixed solution, and separately and simultaneously pumping the first mixed solution and the second mixed solution into a first microreactor of the microchannel reaction device to carry out the first ring-opening reaction to obtain the first reaction solution; and
mixing the second ring-opening reagent with a second organic solvent to obtain a third mixed solution, and separately and simultaneously pumping the third mixed solution and the first reaction solution obtained in step (1) into a second microreactor of the microchannel reaction device to carry out the second ring-opening reaction to obtain the reaction solution comprising the vegetable oil polyol.

In some embodiments, in step (1), the first ring-opening reaction is carried out with a reaction residence time of 3-30 min, preferably 5-15 min, further preferably 11-13 min, even further preferably 12 min.

In some embodiments, in step (1), the first microreactor has a volume of 5 mL-5 L, preferably 5 mL-2 L, further preferably 5 mL-1 L, even further preferably 5-20 mL, most preferably 10 mL.

In some embodiments, in step (2), the second ring-opening reaction is carried out with a reaction residence time of 3-30 min, preferably 5-15 min, further preferably 8-10 min.

In some embodiments, in step (2), the second microreactor has a volume of 5 mL-5 L, preferably 5 mL-2 L, further preferably 5 mL-1 L, even further preferably 5-20 mL, most preferably 10 mL.

In some embodiments, the microchannel reaction device comprises connecting pipelines, a first feed pump, a second feed pump, a third feed pump, a first micromixer, a second micromixer, the first microreactor, the second microreactor, and a receiver; the first feed pump and the second feed pump are connected in parallel to the first micromixer through the pipelines; the first micromixer is connected to the first microreactor; the first microreactor and the third feed pump are connected in parallel to the second micromixer through the pipelines; the second micromixer, the second microreactor, and the receiver are sequentially connected in series through the pipelines;
the connecting pipelines have an inner diameter of 300 μm-10 mm, preferably 300 μm;
the model of the feed pumps is L0107-1A;
the micromixers are Y-type mixers or T-type mixers;
the model of the microreactors is Vapotech, which adopts a microstructured coaxial heat exchanger;
the microreactors are heated using an oil bath pot;
inner pipelines of the microreactors have an inner diameter of 300 μm-10 mm, preferably 300 μm.

In some embodiments, the reaction solution comprising the vegetable oil polyol obtained in step (2) is subjected to a post-treatment, followed by liquid separation; the separated organic phase is neutralized with a base and subjected to liquid separation; the separated organic phase is subjected to rotary evaporation and dried to obtain the vegetable oil polyol.

The vegetable oil polyol prepared by the preparation method described above is also within the protection scope of the present invention.

Specifically, the vegetable oil polyol has a hydroxyl value of 205-285 mg KOH/g, a viscosity of 700-900 mPa-s, and an epoxy value of 0.1%-0.5%.

Use of the vegetable oil polyol described above in the preparation of an antioxidant polyurethane coating material is also within the protection scope of the present invention.

Specifically, the use of the vegetable oil polyol in the preparation of the antioxidant polyurethane coating material comprises the following steps: the vegetable oil polyol prepared by the preparation method described above is subjected to a prepolymerization reaction with an isocyanate compound under the catalysis of an ionic liquid catalyst to obtain a prepolymer mixed solution, then the prepolymer mixed solution is subjected to a polymerization reaction with a chain extender, a flame retardant, and an antioxidant to obtain a polymer mixed solution, and the polymer mixed solution is neutralized with a neutralizing agent and emulsified with deionized water to obtain the antioxidant polyurethane coating material.

In some embodiments, the isocyanate compound is any one or a combination of two or more of toluene diisocyanate, diphenylmethane diisocyanate, 1,6-hexamethylene diisocyanate, and isophorone diisocyanate, preferably isophorone diisocyanate.

In some embodiments, the ionic liquid catalyst is a pyridine-type ionic liquid catalyst, an imidazole-type ionic liquid catalyst, or a long-chain aliphatic amine-type ionic liquid catalyst, preferably any one of the compounds represented by the following structures:

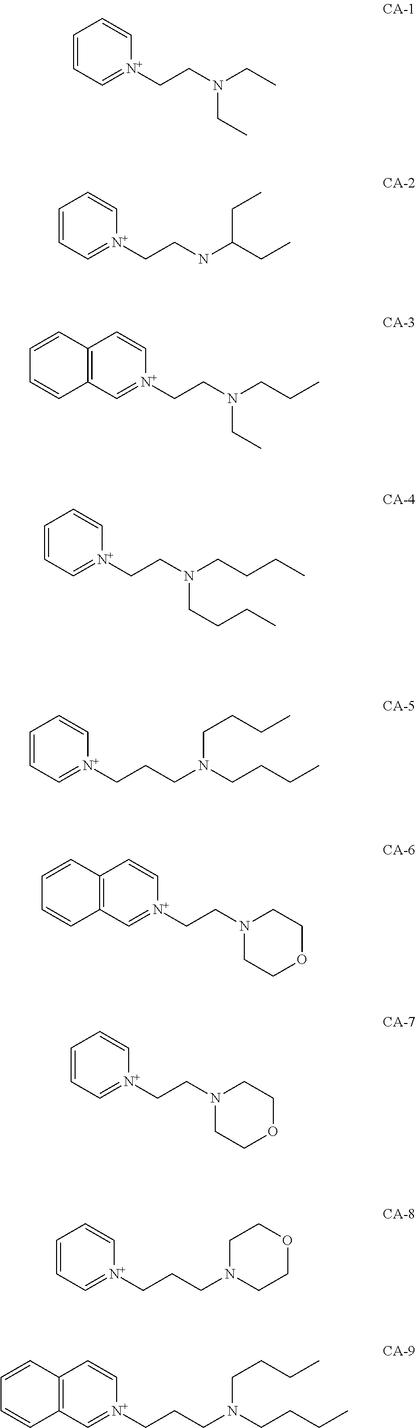

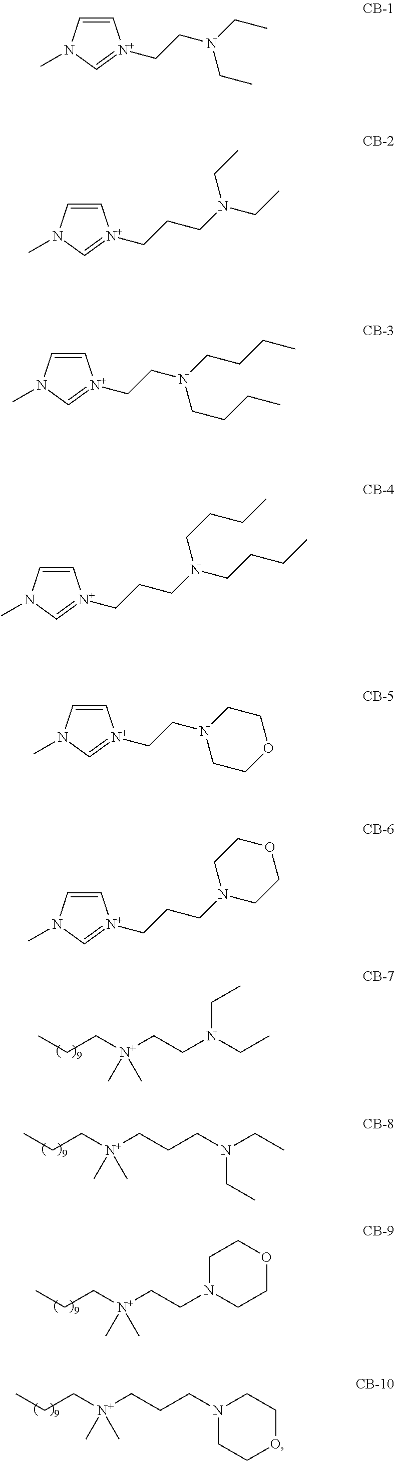

further preferably

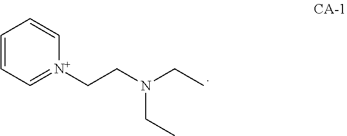

In some embodiments, a mass percentage ratio of the vegetable oil polyol to the ionic liquid catalyst is 1:(0.1%-1%), preferably 1:(0.1%-0.6%), further preferably 1:0.3%.

In some embodiments, the chain extender is any one or a combination of two or more of 1,4-butanediol, ethylene glycol, diethylene glycol, 1,6-hexanediol, hydroquinone bis(2-hydroxyethyl) ether, resorcinol bis(2-hydroxyethyl) ether, bisphenol A bis(2-hydroxyethyl) ether, dimethylolpropionic acid, and dimethylolbutanoic acid, preferably dimethylolpropionic acid; the flame retardant is any one or a combination of two or more of bis(4-hydroxyphenyl)phenylphosphine oxide, tributyl phosphate, and casein, preferably casein; the antioxidant is any one or a combination of two or more of 2,6-di-tert-butyl-p-cresol, antioxidant 1010, Irganox 5057, Naugard PS-30, and a phosphite compound, preferably antioxidant 1010; a structural formula of the phosphite compound is

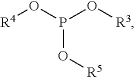

wherein R³, R⁴, and R⁵ are independently selected from phenyl, nonylphenyl, or isodecyl; the neutralizing agent is any one or a combination of two or more of triethylamine, triethanolamine, and dimethylcyclohexylamine, preferably triethylamine;
the dimethylolpropionic acid is abbreviated as DMPA; the 2,6-di-tert-butyl-p-cresol is abbreviated as BHT.

In some embodiments, an anion of the ionic liquid catalyst is a halide anion.

In some embodiments, preferably, the halide anion is a bromide ion or a chloride ion.

In some embodiments, further preferably, the halide anion is a bromide ion.

In some embodiments, a molar ratio of a hydroxyl group in the vegetable oil polyol to a —NCO group in the isocyanate compound is 1:(1.1-1.3), preferably 1:1.2.

In some embodiments, the prepolymerization reaction is carried out at a reaction temperature of 40-100° C., preferably 50° C., for a period of 1-5 h, preferably 2 h.

In some embodiments, a mass ratio of the vegetable oil polyol to the chain extender to the flame retardant to the antioxidant is 1:(0.07-0.11):(0.01-0.1):(0.01-0.1), preferably 1:0.1:0.05:0.05.

In some embodiments, the polymerization reaction is carried out at a reaction temperature of 40-100° C., preferably 50° C., for a period of 1-5 h, preferably 3 h.

In some embodiments, a mass ratio of the vegetable oil polyol to the neutralizing agent is 1:(0.05-0.1), preferably 1:0.09.

In some embodiments, a mass ratio of the vegetable oil polyol to the deionized water is 1:(0.3-0.5), preferably 1:0.4.

In some embodiments, the polymer mixed solution is neutralized with the neutralizing agent until the reaction system is neutral.

In some embodiments, the emulsification process requires shearing at a rotation speed of 1000-14000 rpm for a period of 20-60 min.

### Beneficial Effects

The present invention adopts novel ring-opening reagents to introduce an antioxidant sulfur-containing fragment into the molecular structure of the vegetable oil polyol in a covalent manner, while also introducing a cyclohydrocarbyl group and retaining a small portion of epoxy groups, thereby ensuring that a polyurethane product has a certain degree of toughness, in addition to relatively good corrosion resistance and oxidation resistance, while guaranteeing the mechanical properties of the polyurethane material.

The present invention adopts two specific types of ring-opening reagents to carry out a tandem reaction, such that the prepared vegetable oil polyol has a novel structure, is moderate and uniform in distribution, possesses a relatively low viscosity, and thus can replace traditional petrochemical polyols, and a polyurethane coating material prepared from the vegetable oil polyol prepared by the present invention exhibits significantly improved performance.

### BRIEF DESCRIPTION OF THE DRAWINGS

The present invention will be further illustrated in detail with reference to the following accompanying drawing and detailed description, from which the advantages in the above and/or other aspects of the present invention will become more apparent.

FIG. is a schematic diagram of a microchannel reaction device used in examples of the present invention.

### DETAILED DESCRIPTION

The present invention can be better understood according to the following examples. However, it is easily understood by those skilled in the art that the contents described in the examples are only used to illustrate the present invention, and should not and will not limit the present invention described in detail in the claims.

In the present invention, the related determination methods for the prepared vegetable oil polyols and polyurethane materials are as follows:
the hydroxyl value is determined according to GB/T 12008.3-2009;
the viscosity is determined according to GB/T 12008.7-2010;

the surface drying time of the coating material is determined according to GBRT 1728-2020 (method B);
the hard drying time of the coating material is determined according to GBRT 1728-1979 (method A);
the VOC content is determined according to GBRT 23985-2009 (8.3);
the film hardness by pencil test is determined according to GBRT 6739-2022;
the impact resistance of the coating material is determined according to GBRT 1732-2020;

the oxidation resistance of the coating material is determined according to GBRT 1771-2007;
the 60° gloss of the coating material is determined according to GBRT 9754-2007;
the water resistance of the coating material is determined according to GBRT 1733-1993;
the epoxy value is determined according to GB/T 1677-2008.
the neutral salt spray resistance is determined according to GB/T 1771-2007; and
the flexibility is determined according to GBIT 1731-2020.

FIG. is a schematic diagram of a microchannel reaction device used in examples of the present invention, wherein the microchannel reaction device comprises connecting pipelines, a first feed pump, a second feed pump, a third feed pump, a first micromixer, a second micromixer, the first microreactor, the second microreactor, and a receiver; the first feed pump and the second feed pump are connected in parallel to the first micromixer through the pipelines; the first micromixer is connected to the first microreactor; the first microreactor and the third feed pump are connected in parallel to the second micromixer through the pipelines; the second micromixer, the second microreactor, and the receiver are sequentially connected in series through the pipelines;
the connecting pipelines have an inner diameter of 300 μm;
the model of the feed pumps is L0107-1A;
the micromixers are Y-type mixers;
the model of the microreactors is Vapotech, which adopts a microstructured coaxial heat exchanger;
the microreactors are heated using an oil bath pot;
inner pipelines of the microreactors have an inner diameter of 300 μm.

The reaction solution comprising the vegetable oil polyol obtained in step (2) is subjected to a post-treatment, followed by liquid separation; the separated organic phase is neutralized with a base and then subjected to liquid separation; the separated organic phase is subjected to rotary evaporation and dried to obtain the vegetable oil polyol.

The anion of the ionic liquid catalyst CA-1 used in the examples of the present invention is a bromide ion.

### Example 1

(1) An epoxidized soybean oil (100 g, epoxy value: 6.7%) and an aqueous fluoroboric acid solution (200 mg, 50 wt %) were mixed to obtain a first mixed solution; 1-mercapto-2-propanol (19.4 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.61 mL/min and 0.22 mL/min, respectively, the reaction residence time was maintained at 12 min, and the volume of the first microreactor was 10 mL. After the reaction was completed, a first reaction effluent was obtained.

Cyclopropylmethanol (15.1 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.3 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 249 mg KOH/g, a viscosity of 861 mPa·s, and an epoxy value of 0.1%.

(2) Preparation of vegetable oil-based polyurethane coating material:

The soybean oil polyol (100 g, prepared in this example) was mixed with 59 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 2

(1) An epoxidized cottonseed oil (100 g, epoxy value: 6.0%) and an aqueous fluoroboric acid solution (200 mg, 50 wt %) were mixed to obtain a first mixed solution; 1-mercapto-2-propanol (20.7 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.60 mL/min and 0.23 mL/min, respectively, the reaction residence time was maintained at 12 min, and the volume of the first microreactor was 10 mL. After the reaction was completed, a first reaction effluent was obtained.

Cyclobutylmethanol (12.9 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.29 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a cottonseed oil polyol, with a hydroxyl value of 239 mg KOH/g, a viscosity of 813 mPa·s, and an epoxy value of 0.1%.

(2) Preparation of vegetable oil-based polyurethane coating material:

The cottonseed oil polyol (100 g, prepared in this example) was mixed with 57 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 3

(1) An epoxidized soybean oil (100 g, epoxy value: 6.2%) and an aqueous fluoroboric acid solution (200 mg, 50 wt %) were mixed to obtain a first mixed solution; 1-mercapto-2-butanol (20.6 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.59 mL/min and 0.24 mL/min, respectively, the reaction residence time was maintained at 12 min, and the volume of the first microreactor was 10 mL. After the reaction was completed, a first reaction effluent was obtained.

Cyclopentylmethanol (19.4 g) was dissolved in 50 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.35 mL/min, the reaction residence time was maintained at 8.5 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 230 mg KOH/g, a viscosity of 789 mPa·s, and an epoxy value of 0.2%.

(2) Preparation of vegetable oil-based polyurethane coating material:

The soybean oil polyol (100 g, prepared in this example) was mixed with 55 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 4

(1) An epoxidized cottonseed oil (100 g, epoxy value: 6.0%) and an aqueous fluoroboric acid solution (200 mg, 50 wt %) were mixed to obtain a first mixed solution; 1-mercapto-2-butanol (20 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.59 mL/min and 0.24 mL/min, respectively, the reaction residence time was maintained at 12 min, and the volume of the first microreactor was 10 mL. After the reaction was completed, a first reaction effluent was obtained.

Cyclohexylmethanol (21.4 g) was dissolved in 50 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.35 mL/min, the reaction residence time was maintained at 8.5 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a cottonseed oil polyol, with a hydroxyl value of 223 mg KOH/g, a viscosity of 746 mPa·s, and an epoxy value of 0.3%.

(2) Preparation of vegetable oil-based polyurethane coating material:

The cottonseed oil polyol (100 g, prepared in this example) was mixed with 52 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 5

(1) An epoxidized soybean oil (100 g, epoxy value: 6.5%) and an aqueous fluoroboric acid solution (200 mg, 50 wt %) were mixed to obtain a first mixed solution; 1-mercapto-2-propanol (22.5 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.59 mL/min and 0.24 mL/min, respectively, the reaction residence time was maintained at 12 min, and the volume of the first microreactor was 10 mL. After the reaction was completed, a first reaction effluent was obtained.

Cyclopropylmethanol (14.6 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.29 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 252 mg KOH/g, a viscosity of 823 mPa·s, and an epoxy value of 0.

(2) Preparation of vegetable oil-based polyurethane coating material:

The soybean oil polyol (100 g, prepared in this example) was mixed with 60 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 6

(1) An epoxidized soybean oil (100 g, epoxy value: 6.7%) and an aqueous fluoroboric acid solution (200 mg, 50 wt %) were mixed to obtain a first mixed solution; 1-mercapto-2-propanol (23.2 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.61 mL/min and 0.22 mL/min, respectively, the reaction residence time was maintained at 12 min, and the volume of the first microreactor was 10 mL. After the reaction was completed, a first reaction effluent was obtained.

Cyclobutylmethanol (14.4 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.28 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 259 mg KOH/g, a viscosity of 855 mPa·s, and an epoxy value of 0.2%.

(2) Preparation of vegetable oil-based polyurethane coating material:

The soybean oil polyol (100 g, prepared in this example) was mixed with 62 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 7

(1) An epoxidized cottonseed oil (100 g, epoxy value: 6.0%) and an aqueous fluoroboric acid solution (200 mg, 50 wt %) were mixed to obtain a first mixed solution; 1-mercapto-2-butanol (23.9 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.60 mL/min and 0.23 mL/min, respectively, the reaction residence time was maintained at 12 min, and the volume of the first microreactor was 10 mL. After the reaction was completed, a first reaction effluent was obtained.

Cyclopentylmethanol (15 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.28 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a cottonseed oil polyol, with a hydroxyl value of 230 mg KOH/g, a viscosity of 726 mPa·s, and an epoxy value of 0.2%.

(2) Preparation of vegetable oil-based polyurethane coating material:

The cottonseed oil polyol (100 g, prepared in this example) was mixed with 55 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 8

(1) An epoxidized soybean oil (100 g, epoxy value: 6.5%) and an aqueous fluoroboric acid solution (200 mg, 50 wt %) were mixed to obtain a first mixed solution; 1-mercapto-2-butanol (21.6 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.6 mL/min and 0.23 mL/min, respectively, the reaction residence time was maintained at 12 min, and the volume of the first microreactor was 10 mL. After the reaction was completed, a first reaction effluent was obtained.

Cyclohexylmethanol (23.2 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.28 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 232 mg KOH/g, a viscosity of 763 mPa·s, and an epoxy value of 0.3%.

(2) Preparation of vegetable oil-based polyurethane coating material:

The soybean oil polyol (100 g, prepared in this example) was mixed with 54 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Comparative Example 1

The preparation method for the vegetable oil polyol was the same as that in Example 1, except that the reaction device was a conventional reaction kettle.

(1) An epoxidized soybean oil (100 g, epoxy value: 6.7%) and an aqueous fluoroboric acid solution (200 mg, 50 wt %) were mixed to obtain a first mixed solution; 1-mercapto-2-propanol (19.4 g) was dissolved in 30 mL of dichloromethane to obtain a second mixed solution; cyclopropylmethanol (15.1 g) was dissolved in 40 mL of dichloromethane to obtain a third mixed solution.

The first mixed solution and the second mixed solution were added into the reaction kettle, and the mixture was subjected to a first ring-opening reaction at 65° C. for 4 h. Then, the temperature of the reaction kettle was adjusted to 80° C. The third mixed solution was added to the reaction system, and the mixture was subjected to a second ring-opening reaction for 6 h. After the reaction was completed, the reaction solution was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 85 mg KOH/g, a viscosity of 2239 mPa·s, and an epoxy value of 0.2%.

(2) Preparation of vegetable oil-based polyurethane coating material:

The soybean oil polyol (100 g, prepared in this example) was mixed with 34 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Comparative Example 2

(1) An epoxidized soybean oil (100 g, epoxy value: 6.7%) and an aqueous fluoroboric acid solution (200 mg, 50 wt %) were mixed to obtain a first mixed solution; 1,2-propanediol (16.2 g) was dissolved in 30 mL of dichloromethane to obtain a second mixed solution. After the temperature of the oil bath pot was adjusted to 65° C., the first mixed solution and the second mixed solution were separately and simultaneously pumped into the first microreactor of the microchannel reaction device, and the mixture was subjected to a first ring-opening reaction, wherein the liquid inlet rates of the first mixed solution and the second mixed solution were 0.61 mL/min and 0.22 mL/min, respectively, the reaction residence time was maintained at 12 min, and the volume of the first microreactor was 10 mL. After the reaction was completed, a first reaction effluent was obtained.

Cyclopropylmethanol (15.1 g) was dissolved in 40 mL of dichloromethane to obtain a third mixed solution. After the temperature of the oil bath pot was adjusted to 80° C., the first reaction effluent and the third mixed solution were separately and simultaneously pumped into the second microreactor of the microchannel reaction device, and the mixture was subjected to a second ring-opening reaction, wherein the liquid inlet rate of the third mixed solution was 0.3 mL/min, the reaction residence time was maintained at 9 min, and the volume of the second microreactor was 10 mL. After the reaction was completed, the reaction effluent from the second microreactor was subjected to liquid separation. The separated organic phase was neutralized and washed with a 10 wt % aqueous sodium bicarbonate solution until the pH value was 6.5-7.5, and then subjected to liquid separation. The separated organic phase was subjected to rotary evaporation and dried to obtain a soybean oil polyol, with a hydroxyl value of 255 mg KOH/g, a viscosity of 892 mPa·s, and an epoxy value of 0.1%.

(2) Preparation of vegetable oil-based polyurethane coating material:

The soybean oil polyol (100 g, prepared in this example) was mixed with 61 g of isophorone diisocyanate (IPDI), and 0.3 g of ionic liquid catalyst CA-1 was added. The mixture was then subjected to a prepolymerization reaction at a temperature of 50° C. for 2 h to obtain a prepolymer mixed solution. To the prepolymer mixed solution were added 5 g of antioxidant 1010, 5 g of flame retardant casein, and 10 g of hydrophilic chain extender DMPA. The mixture was then subjected to a polymerization reaction at a temperature of 50° C. for 3 h to obtain a polymer mixed solution. The polymer mixed solution was cooled to 30° C., and then 9 g of neutralizing agent triethylamine was added to neutralize the polymer mixed solution. Deionized water (40 g) was then added, and the mixture was subjected to high-speed shearing emulsification (the shearing rotation speed was 12000 rpm, and the emulsification time was 25 min) to obtain a vegetable oil-based polyurethane coating material.

### Example 9: Product Performance Determination Results

The performance indexes of the vegetable oil polyols prepared in Examples 1-8 and Comparative Examples 1-2 are shown in Table 1, and the performance indexes of the vegetable oil-based polyurethane coating materials prepared in these examples are shown in Table 2.

TABLE 1
Performance indexes of vegetable oil polyols prepared in Examples 1-8 and Comparative Examples 1-2

| | | | | | | | | | Comparative | Comparative |
|---|---|---|---|---|---|---|---|---|---|---|
| Test item | Example 1 | Example 2 | Example 3 | Example 4 | Example 5 | Example 6 | Example 7 | Example 8 | Example 1 | Example 2 |
| Hydroxyl | 249 | 239 | 230 | 223 | 252 | 259 | 230 | 232 | 85 | 255 |
| value (mg | | | | | | | | | | |
| KOH/g) | | | | | | | | | | |
| Viscosity | 861 | 813 | 789 | 746 | 823 | 855 | 726 | 763 | 2239 | 892 |
| (mPa · s) | | | | | | | | | | |

From the data of Comparative Example 1 in Table 1, it is known that the vegetable oil polyol prepared in the conventional reaction kettle had a lower hydroxyl value, but there were more cross-linking side reactions, resulting in a higher viscosity of the vegetable oil polyol; meanwhile, because the reaction in the conventional reaction kettle was uncontrollable, when the first ring-opening reagent and the epoxidized vegetable oil were subjected to the first ring-opening reaction, the hydroxyl groups on the ring-opening reagent could also react with the epoxy groups, leading to the exposure of the mercapto groups.

TABLE 2
Performance indexes of vegetable oil-based polyurethane coating
materials prepared in Examples 1-8 and Comparative Examples 1-2

| | | | | | | | | | Comparative | Comparative |
|---|---|---|---|---|---|---|---|---|---|---|
| Test item | Example 1 | Example 2 | Example 3 | Example 4 | Example 5 | Example 6 | Example 7 | Example 8 | Example 1 | Example 2 |
| Surface | 1 | 1 | 1 | 1 | 1 | 1 | 1 | 1 | 2 | 1 |
| drying | | | | | | | | | | |
| time (h) | | | | | | | | | | |
| Hard | 4 | 4.5 | 5 | 4 | 4 | 5 | 4.5 | 4.5 | 7 | 4.5 |
| drying | | | | | | | | | | |
| time (h) | | | | | | | | | | |
| 60° gloss | 85 | 87 | 92 | 79 | 88 | 83 | 89 | 86 | 68 | 82 |
| Hardness | H | H | H | H | H | H | H | H | F | H |
| by pencil | | | | | | | | | | |
| test | | | | | | | | | | |
| Impact | 50 | 51 | 49 | 52 | 48 | 51 | 48 | 50 | 36 | 42 |
| resistance | | | | | | | | | | |
| (cm) | | | | | | | | | | |
| Oxidation | Passed | Passed | Passed | Passed | Passed | Passed | Passed | Passed | Failed | Failed |
| resistance | | | | | | | | | | |
| Water | 96, | 96, | 96, | 96, | 96, | 96, | 96, | 96, | 48, | 82, |
| resistance | passed | passed | passed | passed | passed | passed | passed | passed | failed | passed |
| (h) | | | | | | | | | | |
| Non- | 99.3 | 99.2 | 99.4 | 99.1 | 98.9 | 99.2 | 99.1 | 99.3 | 98.2 | 99.1 |
| volatile | | | | | | | | | | |
| matter | | | | | | | | | | |
| content | | | | | | | | | | |
| (%) | | | | | | | | | | |
| Flexibility | 1 | 1 | 1 | 1 | 1 | 1 | 1 | 1 | 4 | 2 |
| (mm) | | | | | | | | | | |
| Salt spray | 605 | 623 | 614 | 630 | 623 | 601 | 619 | 632 | 531 | 592 |
| resistance | | | | | | | | | | |
| (h) | | | | | | | | | | |

From Table 2, it can be known that the vegetable oil polyol prepared in the conventional reaction kettle contained exposed hydroxyl groups due to the uncontrollable process, resulting in a decrease in the performance of the vegetable oil-based polyurethane coating material. In addition, due to the introduction of the mercapto groups, the polyurethane coating materials obtained by the present invention had excellent properties such as hardness, impact resistance, oxidation resistance, and the like, making them suitable for industrial production.

The present invention provides an idea and a method for a vegetable oil polyol, a preparation method therefor, and use thereof. There are many methods and ways to specifically implement the technical schemes, and the above description is only of the preferred embodiments of the present invention. It should be noted that those of ordinary skill in the art can make, without departing from the principles of the present invention, various improvements and modifications, and these improvements and modifications shall also be construed within the protection scope of the present invention. All the components not explicitly specified in the examples can be implemented by the prior art.

What is claimed is:

1. A preparation method for a vegetable oil polyol, wherein an epoxidized vegetable oil is subjected to a first ring-opening reaction with an acidic catalyst and a first ring-opening reagent to obtain a first reaction solution, and then the first reaction solution is subjected to a second ring-opening reaction with a second ring-opening reagent to obtain a reaction solution comprising the vegetable oil polyol, wherein
the first ring-opening reagent is a β-mercaptoalcohol compound;
the second ring-opening reagent is a cyclohydrocarbyl methanol compound;
the vegetable oil polyol is prepared using a microchannel reaction device;
the preparation of the vegetable oil polyol using the microchannel reaction device comprises the following steps:
(i) mixing the epoxidized vegetable oil with the acidic catalyst to obtain a first mixed solution, mixing the first ring-opening reagent with a first organic solvent to obtain a second mixed solution, and separately and simultaneously pumping the first mixed solution and the second mixed solution into a first microreactor of the microchannel reaction device to carry out the first ring-opening reaction to obtain the first reaction solution; and
(ii) mixing the second ring-opening reagent with a second organic solvent to obtain a third mixed solution, and separately and simultaneously pumping the third mixed solution and the first reaction solution obtained in step (i) into a second microreactor of the microchannel reaction device to carry out the second ring-opening reaction to obtain the reaction solution comprising the vegetable oil polyol.

2. The preparation method for the vegetable oil polyol according to claim 1, wherein the epoxidized vegetable oil is any one or a combination of two or more of an epoxidized olive oil, an epoxidized peanut oil, an epoxidized rapeseed oil, an epoxidized cottonseed oil, an epoxidized soybean oil, an epoxidized coconut oil, an epoxidized palm oil, an epoxidized sesame oil, an epoxidized corn oil, and an epoxidized sunflower seed oil; the acidic catalyst is any one or a combination of two or more of fluoroboric acid, concentrated sulfuric acid, p-toluenesulfonic acid, methanesulfonic acid, and benzenesulfonic acid; and the acidic catalyst is fluoroboric acid.

3. The preparation method for the vegetable oil polyol according to claim 1, wherein a structural formula of the β-mercaptoalcohol compound is 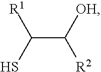
wherein R¹ is selected from hydrogen, methyl, or ethyl; R² is selected from methyl, ethyl, or isopropyl.

4. The preparation method for the vegetable oil polyol according to claim 1, wherein a structural formula of the cyclohydrocarbyl methanol compound is 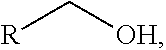
wherein R is selected from cyclopropyl, cyclobutyl, cyclopentyl, cyclohexyl, or cycloheptyl.

5. The preparation method for the vegetable oil polyol according to claim 1, wherein a mass percentage ratio of the epoxidized vegetable oil to the acidic catalyst is 1:(0.02%-0.12%).

6. The preparation method for the vegetable oil polyol according to claim 1, wherein a molar ratio of an epoxy group in the epoxidized vegetable oil to the first ring-opening reagent is 1:(0.4-0.7); and a molar ratio of an epoxy group in the epoxidized vegetable oil to the second ring-opening reagent is 1:(0.3-0.6).

7. The preparation method for the vegetable oil polyol according to claim 1, wherein the first ring-opening reaction and the second ring-opening reaction are carried out at a reaction temperature of 60-100° C., respectively.

8. The preparation method for the vegetable oil polyol according to claim 1, wherein the first organic solvent is any one or a combination of two or more of ethyl acetate, dichloromethane, dichloroethane, chloroform, n-hexane, tetrahydrofuran, 1,4-dioxane, carbon tetrachloride, toluene, and xylene; and the second organic solvent is any one or a combination of two or more of ethyl acetate, dichloromethane, dichloroethane, chloroform, n-hexane, tetrahydrofuran, 1,4-dioxane, carbon tetrachloride, toluene, and xylene.

9. The preparation method for the vegetable oil polyol according to claim 8, wherein a mass-to-volume ratio of the first ring-opening reagent to the first organic solvent is 1 g:0.5-2 mL; and a mass-to-volume ratio of the second ring-opening reagent to the second organic solvent is 1 g:1-4 mL.

10. The preparation method for the vegetable oil polyol according to claim 1, wherein the first ring-opening reaction and the second ring-opening reaction are carried out with a reaction residence time of 3-30 min, respectively.

11. The preparation method for the vegetable oil polyol according to claim 1, wherein the first microreactor has a volume of 5 mL-5 L, and the second microreactor has a volume of 5 mL-5 L.

12. The preparation method for the vegetable oil polyol according to claim 1, wherein the microchannel reaction device comprises connecting pipelines, a first feed pump, a second feed pump, a third feed pump, a first micromixer, a second micromixer, the first microreactor, the second microreactor, and a receiver; the first feed pump and the second feed pump are connected in parallel to the first micromixer through the pipelines; the first micromixer is connected to the first microreactor; the first microreactor and the third feed pump are connected in parallel to the second micromixer through the pipelines; the second micromixer, the second microreactor, and the receiver are sequentially connected in series through the pipelines.

13. A method for utilizing of the vegetable oil polyol of claim 1 comprising a step of adding the vegetable oil polyol in an antioxidant polyurethane coating material.

14. The method according to claim 13, wherein the vegetable oil polyol is subjected to a prepolymerization reaction with an isocyanate compound under the catalysis of an ionic liquid catalyst to obtain a prepolymer mixed solution, then the prepolymer mixed solution is subjected to a polymerization reaction with a chain extender, a flame retardant, and an antioxidant to obtain a polymer mixed solution, and the polymer mixed solution is neutralized with a neutralizing agent and emulsified with deionized water to obtain the antioxidant polyurethane coating material.

15. The method according to claim 14, wherein the isocyanate compound is any one or a combination of two or more of toluene diisocyanate, diphenylmethane diisocyanate, 1,6-hexamethylene diisocyanate, and isophorone diisocyanate.

16. The method according to claim 14, wherein the ionic liquid catalyst is a pyridine-type ionic liquid catalyst, an imidazole-type ionic liquid catalyst, or a long-chain aliphatic amine-type ionic liquid catalyst.

17. The method according to claim 14, wherein a cation of the ionic liquid catalyst is any one of the following structures:

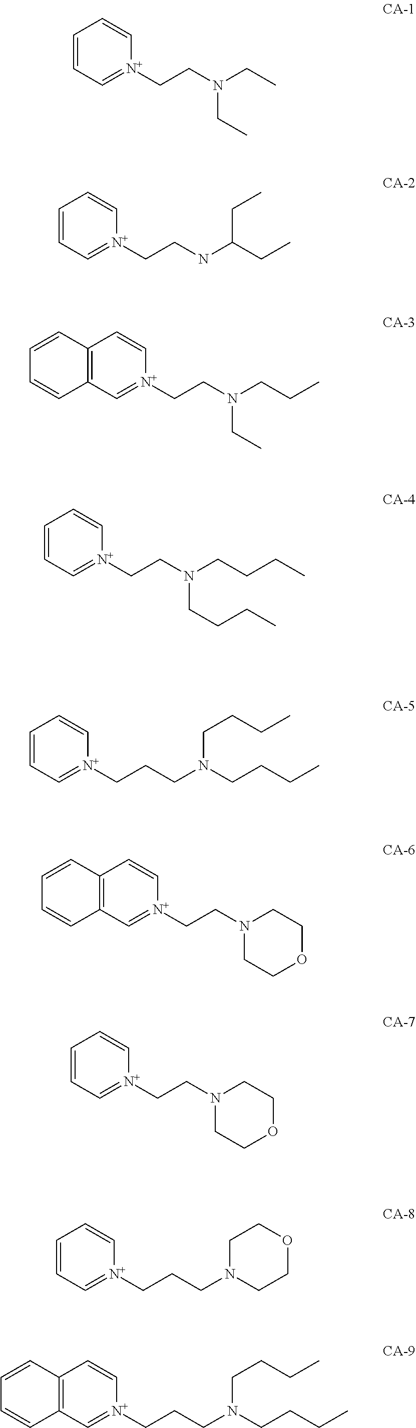

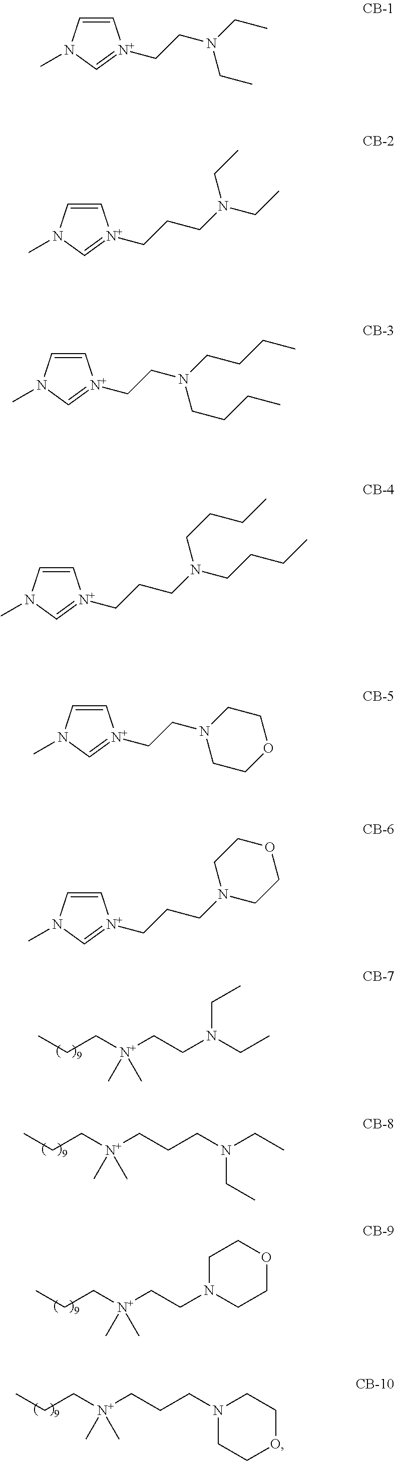

18. The method according to claim 14, wherein a mass percentage ratio of the vegetable oil polyol to the ionic liquid catalyst is 1:(0.1%-1%).

19. The method according to claim 14, wherein the chain extender is dimethylolpropionic acid, or a combination of dimethylolpropionic acid with any one or more of the following components: 1,4-butanediol, ethylene glycol, diethylene glycol, 1,6-hexanediol, hydroquinone bis(2-hydroxyethyl) ether, resorcinol bis(2-hydroxyethyl) ether, bisphenol A bis(2-hydroxyethyl) ether, and dimethylolbutanoic acid;
the flame retardant is any one or a combination of two or more of bis(4-hydroxyphenyl)phenylphosphine oxide, tributyl phosphate, and casein; the antioxidant is any one or a combination of two or more of 2,6-di-tert-butyl-p-cresol, antioxidant 1010, Irganox 5057, Naugard PS-30, and a phosphite compound; a structural formula of the phosphite compound is 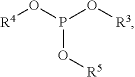
wherein R³, R⁴, and R⁵ are independently selected from phenyl, nonylphenyl, or isodecyl; the neutralizing agent is any one or a combination of two or more of triethylamine, triethanolamine, and dimethylcyclohexylamine.

20. The method according to claim 14, wherein an anion of the ionic liquid catalyst is a halide anion.
